(19) United States
(12) Patent Application Publication
(10) Pub. No.: US 2007/0032660 A1
(43) Pub. Date: Feb. 8, 2007

(54) Purification process for Anastrozole intermediate

(76) Inventors: Alessandro Pontiroli, S. Maria della Versa (PV) (IT); Roberto Casalone, Santhia (VC) (IT)

Correspondence Address: KENYON & KENYON LLP, ONE BROADWAY, NEW YORK, NY, 10004, US

(21) Appl. No.: 11/476,258

(22) Filed: Jun. 27, 2006

Related U.S. Application Data
(60) Provisional application No. 60/694,528, filed on Jun. 27, 2005.

(51) Int. Cl. C07D 249/08; C07C 253/34

(52) U.S. Cl. 548/262.2; 558/409

(57) ABSTRACT

The invention is directed to processes for purifying the Anastrozole intermediate, 3,5-bis(2-cyanoisopropyl)toluene, processes for producing Anastrozole, processes for preparing Anastrozole pharmaceutical compositions, and Anastrozole and Anastrozole pharmaceutical compositions prepared with the processes of the invention.

### RELATED APPLICATIONS

This application claims the benefit of U.S. Provisional Application No. 60/694,528, filed Jun. 27, 2005, herein incorporated by reference.

### FIELD OF THE INVENTION

The present invention relates to a substantially pure intermediate of Anastrozole, 3,5-bis(2-cyanoisopropyl) toluene and purification methods thereof.

### BACKGROUND OF THE INVENTION

Anastrozole, of the chemical name 1,3-benzenediacetonitrile-α,α,α′,α′-tetramethyl-5-(1H-1,2,4-triazole-1-ylmethyl) and having the following chemical structure, 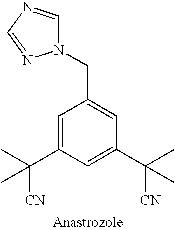
is a potent and selective non-steroidal inhibitor of the aromatase (oestrogen synthetase) system, which converts adrenal androgens to oestrogens in peripheral tissue. It is used in the treatment of advanced or locally advanced breast cancer, and as adjuvant treatment in early breast cancer in postmenopausal women. This drug is available commercially for oral administration ARIMIDEX® by AstraZeneca.

Preparation and purification of Anastrozole was first disclosed in EP 296,749, and comprises: a) the bromination of the toluene derivative, 3,5-bis(2-cyanoisopropyl)toluene in carbon tetrachloride, producing a benzylic bromide; and b) the condensation of the resulting benzylic bromide in dimethylformamide with sodium 1,2,4-triazolyl according to the following scheme: 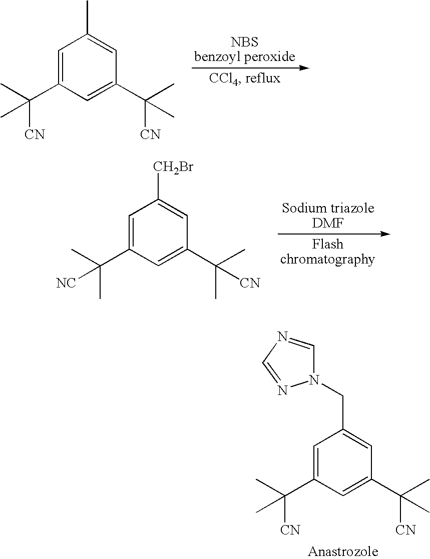
wherein the starting material, 3,5-bis(2-cyanoisopropyl) toluene, of formula I is crystallized from CCl₄, a toxic and carcinogenic solvent, and Anastrozole is obtained after chromatographic separation, a tedious method for industrial scale.

The CHINESE JOURNAL OF MEDICINAL CHEMISTRY, 2003, volume 13, page 146 mentions crystallization of the crude 3,5-bis(2-cyanoisopropyl) toluene of formula I 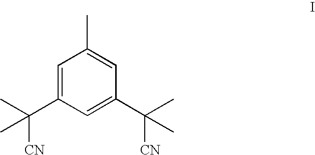
in absolute ethyl alcohol, and using the crystallized intermediate to prepare Anastrozole.

Like any synthetic compound, Anastrozole can contain extraneous compounds or impurities that can come from many sources. They can be unreacted starting materials, by-products of the reaction, products of side reactions, or degradation products. Impurities in Anastrozole or any active pharmaceutical ingredient (API) are undesirable and, in extreme cases, might even be harmful to a patient being treated with a dosage form containing the API.

In addition impurities introduced during commercial manufacturing processes must be limited to very small amounts, and are preferably substantially absent. For example, the ICH Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of an API, such as Anastrozole, it must be analyzed for purity, typically, by HPLC or TLC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and thus, are as safe as possible for clinical use. As discussed above, in the United States, the Food and Drug Administration guidelines recommend that the amounts of some impurities be limited to less than 0.1 percent.

Therefore, additional methods for the purification of 3,5-bis(2-cyanoisopropyl) toluene would be well appreciated.

### SUMMARY OF THE INVENTION

In one aspect, the present invention relates to a process for purifying the Anastrozole intermediate, 3,5-bis(2-cyanoisopropyl)toluene of formula I 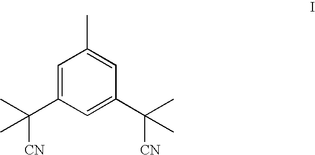
from impurity A of the formula, 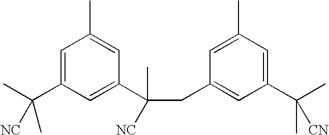
by crystallization from a solvent selected from the group consisting of C₆₋₁₀ aromatic hydrocarbon, and C₃₋₈ ether.

In another aspect, the present invention relates to a process for preparing Anastrozole by purifying the Anastrozole intermediate, 3,5-bis(2-cyanoisopropyl)toluene of formula I, by the process of the present invention, and further converting it to Anastrozole.

In yet another aspect, the present invention further relates to a pharmaceutical composition comprising Anastrozole made by the process of the invention, and pharmaceutically acceptable excipients.

In one aspect, the present invention also relates to a process for preparing pharmaceutical composition comprising mixing Anastrozole made by the process of the invention, and a pharmaceutically acceptable carrier.

### DETAILED DESCRIPTION OF THE INVENTION

Anastrozole prepared comprises a specific impurity, referred to as impurity B, of a proposed structure 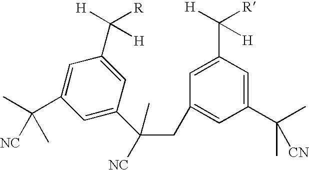
wherein R and R′ can be independently, H or 1,2,4-triazole. This impurity is characterized by an HPLC RRF of 1.35 in relation to Anastrozole. Because this impurity is characterized by a similar solubility to Anastrozole, it is difficult to separate it from Anastrozole. Thus, there is a need in the art for a method of obtaining substantially pure Anastrozole, especially free of impurity B.

The present invention relates to the new discovery that the Anastrozole impurity, impurity B, is derived from an impurity, having an HPLC RRF of 1.53 in relation to Anastrozole, referred to herein as “impurity A” of the structure. 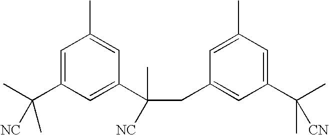
This surprising discovery aided in finding a solution to preparing substantially pure Anastrozole, especially free of impurity B, and preferably free of other impurities, without the use of column chromatographic methods. Also, this method provides Anastrozole with more than 80% yield, preferably more than 90% yield and most preferably more than 95% yield, by purifying 3,5-bis(2-cyanoisopropyl)toluene of formula I, by the utilization of carefully chosen solvents, such as toluene, hence, decreasing the loss of the product, which is greater in polar solvents, such as ethanol that is the solvent of choice in the CHINESE JOURNAL OF MEDICINAL CHEMISTRY, 2003, and also avoiding from using hazardous solvents, such as CCl₄, as used also in EP 296,749.

Specifically, in one aspect of the present invention, a process is presented which involves purifying the Anastrozole intermediate, 3,5-bis(2-cyanoisopropyl)toluene of formula I 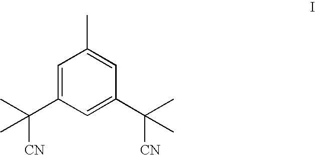
from impurity A of the formula, 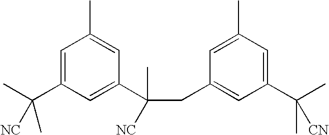
by crystallization from a solvent selected from the group consisting of C₆₋₁₀ aromatic hydrocarbons and C₃₋₈ ethers. The crystallization process typically comprises: providing a solution of 3,5-bis(2-cyanoisopropyl)toluene of formula I in the solvent selected from the group consisting of C₆₋₁₀ aromatic hydrocarbon, and C₃₋₈ Ether; cooling to promote precipitation; and recovering the purified 3,5-bis(2-cyanoisopropyl)toluene of formula I.

The preferred C₆₋₁₀ aromatic hydrocarbon is a C₆₋₈ aromatic hydrocarbon, more preferably, C₆₋₇, and, even most preferably, toluene. Preferably, the C₃₋₈ ether is C₄₋₈, more preferably, C₅₋₈, most preferably, C₅₋₆, and even most preferably either diisopropylether (referred to as DIPE), or methyltertbutylether (referred to MTBE). The more preferred solvent is toluene. Preferably, the solution of 3,5-bis(2-cyanoisopropyl)toluene of formula I is prepared by heating a mixture of the 3,5-bis(2-cyanoisopropyl) toluene of formula I and the solvent. The solvent is preferably used in an amount of from about 2 to about 8 ml of solvent per gram of 3,5-bis(2-cyanoisopropyl)toluene of formula I, more preferably, from about 2.5 to about 4 ml per gram, and, most preferably from about 2.8 to about 3.3 ml per gram. Thus, providing an optimal volume of solvents to obtain a pure product, but also in high yields. Preferably, heating is done to a temperature of about 25° to about 90° C., more preferably of about 50° C. to about 90° C. and most preferably about 60° C. to about 70° C. Preferably, the heating is done to obtain complete dissolution.

Preferably, the cooling stage is done to any temperature cooler than the heating temperature, which will promote precipitation. Typically, cooling is done to a temperature of about 25° C. to about −25° C., preferably, to about 0° C. to about −20° C., and more preferably, to about −10° C. to about −20° C. The cooling may be done in one step or gradually. Preferably, the cooling is done gradually. Preferably, the cooling step includes two stages. Preferably, the first stage includes cooling to a temperature of about 28° C. to about 20° C., more preferably, to 25° C. to about 22° C. Preferably, the second stage includes cooling to a temperature of about 0° C. to about −20° C. Preferably, the first cooling stage is done over a period of about 1 to about 6 hours, more preferably, for about 1 to about 2 hours, and even more preferably, for about 60 minutes to about 70 minutes. Preferably, the second cooling stage is done over a period of about 1 to 3 hours, more preferably, for about 1 to about 2 hours. Preferably, a suspension is obtained when cooling. Preferably, after the cooling process ends, the suspension is maintained for about 30 minutes to about 90 minutes, more preferably, for about 60 to about 90 minutes.

Recovery of the substantially pure 3,5-bis(2-cyanoisopropyl)toluene of formula I may be by conventional techniques, preferably, by filtration.

Preferably, each crystallization results in at least a 25% decrease in the amount of impurity A, preferably more than 40% and most preferably, more than 50%. The process may be repeated until the desired purity is obtained. Thus, the process of the invention can further comprise analyzing the 3,5-bis(2-cyanoisopropyl)toluene of formula I with HPLC, after each crystallization and repetition of crystallization process when necessary.

Preferably, the amount of impurity A present after purification is not more than 0.10 HPLC area percent, preferably, not more than about 0.06 HPLC area percent. In addition, the process to obtain substantially pure 3,5-bis(2-cyanoisopropyl)toluene, preferably, reduces the content of any single impurity present to an amount of less than 0.10 HPLC area percent.

The present invention relates to a process for preparing Anastrozole by purifying the Anastrozole intermediate, 3,5-bis(2-cyanoisopropyl)toluene of formula I by the process of the present invention, and further converting it to Anastrozole.

The synthesis may be done, for example, according to the method disclosed in Co-application No. 60/669,132.

The method comprises: combining 3,5-bis (2-cyanoisopropyl)toluene of formula I, 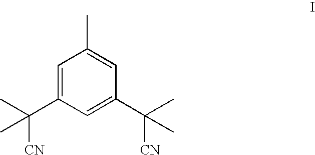
a solvent selected from the group consisting of acetonitrile (referred to as ACN), dichloromethane (referred to as DCM) and chlorobenzene, a brominating reagent selected from the group consisting of N-bromosuccinimide (referred to as NBS) and 1,3-dibromo-5,5-dimethylhydantoin, and 2,2′-azobis(2-methylpropionitrile); heating; combining with 1,2,4-triazole, a solvent selected from the group consisting of N-methylpyrrolidine (referred to as NMP), dimethylformamide (referred to as DMF), mixtures of NMP and DMF, dimethylsulfoxide (referred to as DMSO), mixtures of DMSO and toluene, acetone, ACN, and tetrahydrofuran (referred to as THF), a base selected from the group consisting of NaOH, KOH, K₂CO₃, and Na₂CO₃, and 1,3-benzendiacetonitrile-5-(bromomethyl)-α,α,α′,α′-tetramethyl of formula II, 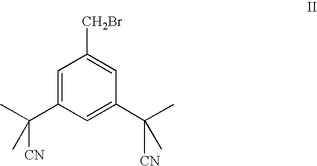
at a temperature below −20° C.; extracting with a mixture comprising of toluene, a linear, branched, or cyclic C₅₋₈ hydrocarbon and water; adding water; extracting the aqueous phase using toluene; extracting the organic phase with a polar mixture containing a solvent selected from the group consisting of NMP and C₁₋₃ alcohols mixed with water, and adding a linear, branched, or cyclic C₅₋₈ hydrocarbon to the organic phase to precipitate Anastrozole.

Preferably, substantially pure Anastrozole is obtained by using substantially pure 3,5-bis(2-cyanoisopropyl)toluene of formula I. Preferably, the substantially pure Anastrozole has a purity greater than 99.9% area by HPLC. More preferably, the substantially pure Anastrozole comprises impurity B in an amount of no more than 0.06% HPLC purity.

The present invention further relates to a pharmaceutical composition comprising Anastrozole made by the process of the invention, and pharmaceutically acceptable excipients.

The present invention also relates to a process for preparing pharmaceutical composition comprising mixing Anastrozole made by the process of the invention, and a pharmaceutically acceptable carrier.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention. The examples set forth below describe single crystallization experiments, which can be repeated to obtain the same yields and improvements in purification until the final desired purity is obtained.

### EXAMPLES

Instrumentation

Column & Packing: HYPERSIL BDS C18; 3 μm, 100 mm×4.6 mm, cat n. 28103-104630 or equivalent

| Eluent A: | Water | | |
|---|---|---|---|
| Eluent B: | Acetonitrile | | |
| Gradient | Time (min) | % Eluent A | % Eluent B |
| | 0 | 80 | 20 |
| | 30 | 40 | 60 |
| | 35 | 20 | 80 |
| | 36 | 80 | 20 |

| | |
|---|---|
| Stop time: | 35 minutes |
| Equilibrium time: | 5 minutes |
| Flow Rate: | 1.0 ml/mins. |
| Detector: | UV at 210 nm |
| Column temperature: | 60° C. |
| Injection: | 5 μl |
| Diluent: | Acetonitrile |

The Mobile phase composition and flow rate may be varied in order to achieve the required system suitability.

### Example 1

### Crystallization of 3,5-bis(2-cyanoisopropyl)toluene From 2 Volumes of Toluene

A 2.5 g sample of 3,5-bis(2-cyanoisopropyl)toluene, having an initial impurity A content of 1.10 HPLC area percent, was suspended in 5 ml of toluene, and heated to 45° C., until complete dissolution occurred. The solution was then allowed to cool to 25° C. over a period of 1 hour, obtaining a suspension, and after 30 minutes at 25° C., the resulting suspension was filtered, and the filtrate was rinsed with 2.5 ml of toluene that was pre-cooled to 0° C. Purified 3,5-bis(2-cyanoisopropyl)toluene was recovered in an amount of 2.1 g, having an impurity A content of 0.46 HPLC area percent.

### Example 2

### Crystallization of 3,5-bis(2-cyanoisopropyl)toluene From 2.5 Volumes of Toluene

A 4 g sample of 3,5-bis(2-cyanoisopropyl)toluene, having an initial impurity A content of 1.93 HPLC area percent, was suspended in 10 ml of toluene, and heated to 65° C., until complete dissolution occurred. The solution was then allowed to cool to 25° C. over a period of 1 hour obtaining a suspension, and then cooled to 0° C. over a period of 2 hours. After 30 min at 0° C., the resulting suspension was filtered, and the filtrate was rinsed with 2.5 ml of toluene, pre-cooled to 0° C. Purified 3,5-bis(2-cyanoisopropyl)toluene was recovered in an amount of 3.2 g, having an impurity A content of 1.02 HPLC area percent.

### Example 3

### Crystallization of 3,5-bis(2-cyanoisopropyl)toluene From 3 Volumes of Toluene

A 42 g sample of 3,5-bis(2-cyanoisopropyl)toluene, having an initial impurity A content of 0.11 HPLC area percent was suspended in 130 ml of toluene, and heated to 61° C., until complete dissolution occurred. The solution was then allowed to cool to 25° C. over a period of 3 hours obtaining a suspension, and then cooled to −20° C. over a period of 2 hours. After 30 min at −20° C., the resulting suspension was filtered, and the filtrate was rinsed with 2.5 ml of toluene that was pre-cooled to −20° C. Purified 3,5-bis(2-cyanoisopropyl)toluene was recovered in an amount of 40.1 g, having an impurity A content of 0.06 HPLC area percent.

### Example 4

### Crystallization and Recrystallization of 3,5-bis(2-cyanoisopropyl)toluene From Toluene

3,5-bis (2-cyanoisopropyl)toluene (50 g), containing 0.45% of impurity A, was dissolved in toluene (150 ml) and heated to 65-70° C. until a complete solution was obtained. After 10 min, the solution was then allowed to cool to 25° C. in 6 h. After this time, the suspension was cooled to −20° C. in 1 hour, stirred at the same temperature for 30 min and then filtered. The solid was then washed with toluene (25 ml) pre-cooled to −20° C.

The wet solid was then analyzed via HPLC showing a content of 0.24% of impurity A. Recrystallizing this solid two more times gave 3,5-bis (2-cyanoisopropyl)toluene having 0.07% of impurity A. this solid was then dried in oven at 50° C. until all solvent were removed.

### Example 5

### Crystallization From 6 Volumes of Ethanol

A 2.5 g sample of 3,5-bis(2-cyanoisopropyl)toluene, having an initial impurity A content of 1.10 HPLC area percent, was suspended in 15 ml of ethanol, and heated to reflux. The solution was then allowed to cool to 25° C. over a period of 3 hours obtaining a suspension, and then filtered. The filtrate is rinsed with 2.5 ml of ethanol, pre-cooled to 0° C. Purified 3,5-bis(2-cyanoisopropyl)toluene is recovered in an amount of 3.2 g, having an impurity A content of 0.61 HPLC area percent.

### Example 6

### Crystallization From DIPE

1.0 g of 3,5-bis(2-cyanoisopropyl)toluene (containing 1.93% of impurity A) was suspended in 10 ml of diisopropyl ether (DIPE), and heated to reflux for 5 hours to obtain a solution. The solution was then cooled to 25° C. over one hour, obtaining a suspension, which was the filtered to give 880 mg of 3,5-bis(2-cyanoisopropyl)toluene containing 1.67% of impurity A.

### Example 7

### Crystallization From MTBE

1.0 g of 3,5-bis(2-cyanoisopropyl)toluene (containing 1.12% of impurity A) was suspended in 10 ml of MTBE, and heated to reflux for 5 hours to obtain a solution. The solution was then cooled to 25° C. over one hour, obtaining a suspension, which was filtered to give 840 mg of 3,5-bis(2-cyanoisopropyl)toluene containing 0.71% of impurity A.

### Example 8

### Synthesis of Anastrozole [1-bromo-3,5-bis(2-cyanoisopropyl)toluene]

A: Formation of 1-bromo-3,5-bis(2-cyanoisopropyl)toluene

A 30 g sample of the 3,5-bis (2-cyanoisopropyl)toluene having 0.06% area by HPLC of impurity A, (produced in example 3) was dissolved in 150 ml of acetonitrile, and 24.8 g of N-bromosuccinimide were added. The resulting suspension was heated to 50° C. for 30 minutes, until a light yellow solution was obtained. Then, 0.5 g of 2,2′-azobis(2-methylpropionitrile) was added, and the reaction was heated to 70° C. for 6 hours. The solution was then allowed to cool to 20° C., and poured into 150 ml of a 5 percent by weight solution of sodium metabisulphite in water with vigorous stirring. The organic layer was then separated and washed with 100 ml of a 5 percent by weight solution of sodium carbonate in water before removing the organic solvent under reduced pressure, until a total volume of 90 ml was obtained. The resulting slurry was then heated to 50° C., and 150 ml of heptane were slowly added over a period of 30 minutes, raising the temperature to 70° C. The suspension was then allowed to cool to 20° C., and filtered on a sintered glass funnel. Drying under reduced pressure yields 54 g of crude 1-bromo-3,5-bis(2-cyanoisopropyl)toluene in 85 percent purity (HPLC).

B: Formation of Anastrozole

A 16.7 g sample of 1,2,4-triazole was dissolved in 52 ml of NMP at 20° C., and 9.7 g of NaOH was added in portions over 1 hour, while maintaining the temperature at less than 35° C. The solution was stirred for 18 hours at 20° C., and then cooled to −30° C. A solution of 40 g of crude alpha-bromo-3,5-bis(2-cyanoisopropyl)toluene in 60 ml of NMP was slowly added over 6 hours, while maintaining the temperature below −20° C.

At the end of the addition, the suspension was stirred for 18 hours at −20° C., and, during that time, the reaction was monitored via HPLC. When the amount of starting material was less than 0.5 percent, acetic acid was added in an amount sufficient to provide a pH of about 6.5 to about 7. The mixture was slowly allowed to warm to 20° C., then 120 ml of toluene, 240 of heptane, and 170 ml of water were added. The biphasic system was stirred vigorously for 30 minutes, and the organic layer was then separated. Then, 240 ml of water, 60 ml of toluene, and 120 ml of heptane were added to the aqueous phase, and the system was stirred for 30 minutes before the organic phase was separated. Then, 400 ml of toluene and 240 ml of water were added to the aqueous portion, and the biphasic system was stirred for 1 hour. The organic layer was separated, and washed 3 times with 180 ml of a 0.05N solution of sulphuric acid in water. The final organic phase was concentrated under reduced pressure to a final volume of 150 ml at 40° C., and 180 ml of heptane were added drop-wise over a period of 1 hour. The suspension was cooled to 0° C., stirred for 1 hour, and filtered. The crude solid was dissolved in 390 ml of 2-propanol at 50° C., and 78 ml of heptane were slowly added under stirring.

The solution was cooled to 0° C., stirred for 1 hour, and filtered. The solid was dried at 55° C. under reduced pressure until a constant weight was achieved; producing 23.5 g of product with a purity of 99.94 HPLC area percent having 0.06% of impurity B, and a melting point of 85° C., as measured by DSC.

What is claimed:

1. A process for purifying Anastrozole intermediate, 3,5-bis(2-cyanoisopropyl)toluene of formula I 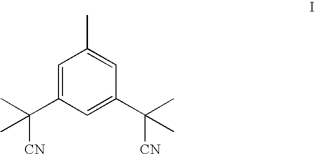
from impurity A of the formula, 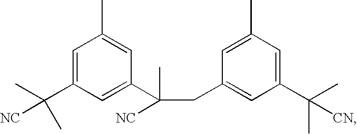
comprising crystalizing the 3,5-bis(2-cyanoisopropyl)toluene from a solvent selected from the group consisting of C₆₋₁₀ aromatic hydrocarbons and C₃₋₈ ethers.

2. The process of claim 1, wherein said crystallization comprises:
providing a solution of 3,5-bis(2-cyanoisopropyl)toluene of formula I in the solvent selected from the group consisting of C₆₋₁₀ aromatic hydrocarbons and C₃₋₈ ethers;
cooling to promote precipitation; and
recovering the purified 3,5-bis(2-cyanoisopropyl)toluene of formula I.

3. The process of claim 2, wherein the C₆₋₁₀ aromatic hydrocarbon is a C₆₋₈ aromatic hydrocarbon.

4. The process of claim 3, wherein the C₆₋₈ aromatic hydrocarbon is a C₆₋₇ aromatic hydrocarbon.

5. The process of claim 4, wherein the C₆₋₇ aromatic hydrocarbon is toluene.

6. The process of claim 2, wherein the C₃₋₈ ether is a C₄₋₈ ether.

7. The process of claim 6, wherein the C₄₋₈ ether is a C₅₋₈ ether.

8. The process of claim 7, wherein the C₅₋₈ ether is a C₅₋₆ ether.

9. The process of claim 8, wherein the C₅₋₆ ether is either diisopropylether or methyltertbutylether.

10. The process of claim 2, wherein the solvent is toluene.

11. The process of claim 2, wherein the solution, in step a, is prepared by heating a mixture of the 3,5-bis(2-cyanoisopropyl) toluene of formula I and the solvent.

12. The process of claim 2, wherein the solvent, in step a, is used in an amount of from about 2 to about 8 ml per gram of 3,5-bis(2-cyanoisopropyl)toluene of formula I.

13. The process of claim 2, wherein the the solvent, in step a, is used in an amount from about 2.5 to about 4 ml per gram of 3,5-bis(2-cyanoisopropyl)toluene of formula I.

14. The process of claim 13, wherein the the solvent, in step a, is used in an amount from about 2.8 to about 3.3 ml per gram of 3,5-bis(2-cyanoisopropyl)toluene of formula I.

15. The process of claim 11, wherein the heating is done to a temperature of about 25° to about 90° C.

16. The process of claim 2, wherein the cooling, in step b, is done to a temperature of about 25° C. to about −25° C.

17. The process of claim 16, wherein the cooling includes first and second stages.

18. The process of claim 17, wherein the first stage includes cooling to a temperature of about 28° C. to about 20° C.

19. The process of claim 17, wherein the second stage includes cooling to a temperature of about 0° C. to about −20° C.

20. The process of claim 17, wherein the first cooling stage is done over a period of about 1 to about 6 hours.

21. The process of claim 19, wherein the second cooling stage is done over a period of about 1 to 3 hours.

22. The process of claim 2, wherein a suspension is obtained when cooling.

23. The process of claim 22, wherein step b further comprises maintaining the suspension for about 30 minutes to about 90 minutes.

24. The process of claim 2, wherein each crystallization results in at least a 25% decrease in the amount of impurity A.

25. The process of claim 24, wherein each crystallization results in a more than 40% decrease in the amount of impurity A.

26. The process of claim 25, wherein each crystallization results in a more than 50% decrease in the amount of impurity A.

27. The process of claim 2, wherein the amount of impurity A present after purification is not more than 0.10 HPLC area percent.

28. The process of claim 2, wherein the amount of impurity A present after purification is not more than about 0.06 HPLC area percent.

29. The process of claim 2, wherein the content of any single impurity present after purification is less than 0.10 HPLC area percent.

30. The process of claim 1, further comprising converting the purified 3,5-bis(2-cyanoisopropyl)toluene of formula I to Anastrozole.

31. The process of claim 30, further comprising the steps of:
(a) combining 3,5-bis (2-cyanoisopropyl)toluene of formula I, 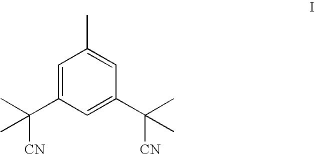
a solvent selected from the group consisting of acetonitrile, dichloromethane and chlorobenzene, a brominating reagent selected from the group consisting of N-bromosuccinimide and 1,3-dibromo-5,5-dimethylhydantoin, and 2,2′-azobis(2-methylpropionitrile);
(b) heating;
(c) combining with 1,2,4-triazole, a solvent selected from the group consisting of N-methylpyrrolidine, dimethylformamide, mixtures of NMP and DMF, dimethylsulfoxide, mixtures of DMSO and toluene, acetone, ACN, and tetrahydrofuran, a base selected from the group consisting of NaOH, KOH, K₂CO₃, and Na₂CO₃, and 1,3-benzendiacetonitrile-5-(bromomethyl)-α,α,α′,α′- tetramethyl of formula II, 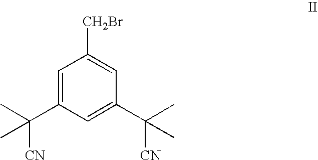
at a temperature below −20° C.;
(d) extracting with a mixture comprising of toluene, linear, branched or cyclic C₅₋₈ hydrocarbon and water;
(e) adding water;
(f) extracting the aqueous phase using toluene;
(g) extracting the organic phase with a polar mixture containing a solvent selected from the group consisting of NMP and C₁₋₃ alcohol mixed with water, and
(h) adding linear, branched or cyclic C₅₋₈ hydrocarbon to the organic phase to precipitate Anastrozole.

32. The process of claim 31, wherein substantially pure Anastrozole is obtained.

33. The process of claim 32, wherein the substantially pure Anastrozole is in purity greater than 99.9% area by HPLC.

34. The process of claim 32, wherein the substantially pure Anastrozole comprises impurity B in an amount of no more than 0.06% HPLC purity.

35. The process of claim 33, wherein the substantially pure Anastrozole comprises impurity B in an amount of no more than 0.06% HPLC purity.

36. A pharmaceutical composition comprising the Anastrozole prepared with the process of claim 33 and pharmaceutically acceptable excipients.

37. A pharmaceutical composition comprising the Anastrozole prepared with the process of claim 34 and pharmaceutically acceptable excipients.

38. A process for preparing pharmaceutical composition comprising mixing the Anastrozole prepared with the process of claim 33 and a pharmaceutically acceptable carrier.

39. A process for preparing pharmaceutical composition comprising mixing the Anastrozole prepared with the process of claim 34 and a pharmaceutically acceptable carrier.
